SEQUENCE LISTING (14 sequences)

SEQ ID NO: 1 - DNA, 1860 bp, Sphingobacterium multivorum
  CDS (1)..(1860)
atgaaaaatacaatttcgtgcctaactttagcgcttttaagcgcaagccagttacatgctcaaacagctgccgactcggcttatgttagagatcattatgaaaagaccgaagtagcaattcccatgcgagatgggaaaaaattatttactgcgatctacagtccaaaagacaaatccaagaaatatccagttttgctcaatagaacgccctacacggtttctccttatgggcagaacgaatacaaaaaaagtttgggaaactttccccaaatgatgcgtgaaggctatattttcgtttaccaggatgtccgtggcaagtggatgagcgaaggtgattttgaagatatacgtccgaccacgtacagcaaagataaaaaagcaatcgatgaaagtacggatacctatgatgcgcttgaatggttacagaaaaatctcaaaaactataatggcaaagccgggctctatgggatttcctatccaggcttctattctaccgtcggattggtcaaaacacacccgagcttgaaggcagtctccccacaggctcccgtaacagactggtatatcggcgacgacttccaccataatggcgtattgtttcttcaggatgcatttacattcatgtcaacctttggtgtccctcgtccaaaacccattacaccggatcaatttaagggcaaaattcagatcaaagaagccgataaatataacttttttgcagaagcaggaacagcgcgggaactcaaagaaaaatactttggtgactccgtacaattttggaatgacctgtttaagcatcccgactatgatgatttttggaaatcgcgtgtgatcaccaattctttacaggaggtaaaaccagctgtgatggtggttggtggtttctttgacgcggaagatgcttatggaacatttaagacctaccaatcgattgaggataaaagcaaaaaaaacaactcgattttagtcgcgggaccttggtatcatggcggctgggttcgtgcagaaggaaactatttaggtgatatccaatttgagaaaaaaaccagtattacttatcaggaacaatttgaacaaccgtttttcaaatattacctaaaagatgaaggaaacttcgccccttccgaagccaacatttttgtttcaggcagcaacgaatggaaacatttcgaacaatggccaccaaaaaatgtagagacaaaaaaactatacttccaacctcaggggaaacttggatttgacaaagttcaacgtacagattcctgggatgaatatgtaacagacccgaataaacctgttccgcatcaaggtgggttaattcaaaaccgaacacgggagtatatggtagatgatcaacgtttcgcggctagtcgccctgatgtcatggtttatcaaacggaaccgttgacggaggacctgacgatagtaggcccaatcaaaaactttctcaaagtttcttcaacaggaacagacgcggactatgttgtcaaactgattgacgtttatccgaatgatgcagcaagttatcaaggaaaaacaatggctggatatcaaatgatggtacgtggtgagatcatggcggggaaataccgaaatggtttcgataaagcgcaggccttgactccaggtatggtcgaaaaggtgaattttgaaatgccagacgttgcgcataccttcaaaaaaggacatcgcattatggttcaggtacaaaactcatggtttccgctggcagaacgaaatccacaggtgtttttagcaccttatacagctaccaaagctgatttccgcaaagctacccaacgtatttttcacgatgtgaacaatgccacatacatcgaattttctgtcctcaaagattag

SEQ ID NO: 2 - protein, 619 aa, Sphingobacterium multivorum
MKNTISCLTLALLSASQLHAQTAADSAYVRDHYEKTEVAIPMRDGKKLFTAIYSPKDKSKKYPVLLNRTPYTVSPYGQNEYKKSLGNFPQMMREGYIFVYQDVRGKWMSEGDFEDIRPTTYSKDKKAIDESTDTYDALEWLQKNLKNYNGKAGLYGISYPGFYSTVGLVKTHPSLKAVSPQAPVTDWYIGDDFHHNGVLFLQDAFTFMSTFGVPRPKPITPDQFKGKIQIKEADKYNFFAEAGTARELKEKYFGDSVQFWNDLFKHPDYDDFWKSRVITNSLQEVKPAVMVVGGFFDAEDAYGTFKTYQSIEDKSKKNNSILVAGPWYHGGWVRAEGNYLGDIQFEKKTSITYQEQFEQPFFKYYLKDEGNFAPSEANIFVSGSNEWKHFEQWPPKNVETKKLYFQPQGKLGFDKVQRTDSWDEYVTDPNKPVPHQGGLIQNRTREYMVDDQRFAASRPDVMVYQTEPLTEDLTIVGPIKNFLKVSSTGTDADYVVKLIDVYPNDAASYQGKTMAGYQMMVRGEIMAGKYRNGFDKAQALTPGMVEKVNFEMPDVAHTFKKGHRIMVQVQNSWFPLAERNPQVFLAPYTATKADFRKATQRIFHDVNNATYIEFSVLKD

SEQ ID NO: 3 - DNA, 33 bp, Artificial
  primer
catgccatggatcaaacagctgccgactcggct

SEQ ID NO: 4 - DNA, 31 bp, Artificial
  primer
ccgctcgagctaatctttgaggacagaaaat

SEQ ID NO: 5 - DNA, 35 bp, Artificial
  primer
gggaattccatatgcaaacagctgccgactcggct

SEQ ID NO: 6 - DNA, 34 bp, Artificial
  primer
gggaattccatatgaaaaatacaatttcgtgcct

SEQ ID NO: 7 - DNA, 33 bp, Artificial
  primer
gggaattccatatgcatgctcaaacagctgccg

SEQ ID NO: 8 - DNA, 34 bp, Artificial
  primer
gggaattccatatggctcaaacagctgccgactc

SEQ ID NO: 9 - DNA, 36 bp, Artificial
  primer
gggaattccatatgacagctgccgactcggcttatg

SEQ ID NO: 10 - DNA, 35 bp, Artificial
  primer
gggaattccatatggctgccgactcggcttatgtt

SEQ ID NO: 11 - DNA, 35 bp, Artificial
  primer
gggaattccatatggccgactcggcttatgttaga

SEQ ID NO: 12 - DNA, 35 bp, Artificial
  primer
gggaattccatatggactcggcttatgttagagat

SEQ ID NO: 13 - DNA, 35 bp, Artificial
  primer
gggaattccatatgtcggcttatgttagagatcat

SEQ ID NO: 14 - DNA, 35 bp, Artificial
  primer
gggaattccatatggcttatgttagagatcattat

(19) United States
(12) Patent Application Publication
(10) Pub. No.: US 2011/0262962 A1
(43) Pub. Date: Oct. 27, 2011

(54) METHOD FOR PRODUCING PEPTIDES

(75) Inventors: ISAO ABE, Kanagawa (JP); NOBUHIRO HIRATSUKA, Kanagawa (JP); SONOKO TAKEUCHI, Kanagawa (JP); SHUNICHI SUZUKI, Kanagawa (JP); KENZO YOKOZEKI, Kanagawa (JP)

(73) Assignee: AJINOMOTO CO., INC., TOKYO (JP)

(21) Appl. No.: 13/087,612

(22) Filed: Apr. 15, 2011

(30) Foreign Application Priority Data
Apr. 15, 2010 (JP) 2010-094407

(51) Int. Cl. C12P 21/02

(52) U.S. Cl. 435/68.1

(57) ABSTRACT

The present invention provides a method for producing a peptide, comprising culturing a transformant introduced with an expression vector to prepare a culture, and mixing the culture with a carboxy component and an amine component to form the peptide. The expression vector comprises a polynucleotide encoding a protein: (A) having selected deletions in the amino acid sequence of SEQ ID NO:2, (B) having a mutation of one or several amino acid residues in any protein selected from said group (A); (C) having 70% or more amino acid sequence identity to any protein selected from said group (A), (D) encoded by a polynucleotide that hybridizes under a stringent condition with a polynucleotide consisting of a nucleotide sequence complementary to a polynucleotide encoding any protein selected from said group (A), and (E) encoded by a polynucleotide having 70% or more nucleotide sequence identity to the polynucleotide encoding any protein selected from the group (A).

### TECHNICAL FIELD

The present invention relates to a method for producing a peptide, and particularly relates to a method for producing the peptide and a method for producing proteins to be used for producing the peptide.

### BACKGROUND ART

Peptides are utilized in various fields such as pharmaceuticals and foods. For example, L-alanyl-L-glutamine is more stable and more soluble in water than L-glutamine, and thus, is widely used as a component of infusion solutions and serum-free media.

Chemical synthesis methods have been conventionally known as the method for producing the peptide, but these methods are not always satisfactory in terms of easiness and efficiency.

On the other hand, methods for producing the peptide using an enzyme have been developed (e.g., EP 278787 A1 and EP 359399 B1). However, in the conventional methods for producing the peptide using the enzyme, there has been room for improvement in that a peptide forming rate is extremely slow and a peptide forming yield is low. Under such a context, it is desired to develop a method for industrially producing the peptide with high efficiency. As one of its measures, it has been attempted to unearth and ameliorate enzymes suitable for the industrial production of the peptide.

For the enzyme that is excellent in peptide forming activity, an enzyme derived from Sphingobacterium has been found (e.g., WO 2004/011653, JP 2005-058212-A and WO 2006/075486).

### DISCLOSURE OF INVENTION

### Problem to be Solved by the Invention

It is an object of the present invention to provide a method for producing a peptide with high efficiency.

### Means for Solving Problem

The present inventors focused on amino acid ester transpeptidase (hereinafter, the amino acid ester transpeptidase may be abbreviated as AET) derived from Sphingobacterium multivorum (hereinafter, Sphingobacterium multivorum may be abbreviated as S. multivorum) as an enzyme used for the method for producing the peptide. According to sequence prediction in silico for the N terminus of AET derived from S. multivorum, it was estimated that the amino acid sequence from the methionine residue at position 1 to the alanine residue at position 20 in the amino acid sequence of SEQ ID NO:2 was deduced to encode a signal peptide. Thus, generally thinking, AET derived from S. multivorum was expected to be localized in periplasm.

The present inventors studied the localization of AET derived from S. multivorum that was expressed in Escherichia coli (hereinafter, Escherichia coli may be abbreviated as E. coli), and unexpectedly found that both soluble AET and insoluble AET were localized in cytosol and the signal peptide was cleaved in both the cases. That is, it was speculated that although the signal peptide of AET derived from S. multivorum was cleaved when expressed in E. coli, the signal peptide did not so contribute to translocation of AET to the periplasm. Such a phenomenon is not common as the protein having the signal peptide.

Furthermore, the present inventors prepared the AET-expressing strain in which the N terminal signal peptide of AET derived from S. multivorum had been substituted with one derived from Erwinia carotovora and AET-expressing strains in which the N terminal signal peptide had been deleted at various positions, and carried out various experiments in order to determine which is more suitable the cytosol or the periplasm for the localization of AET derived from S. multivorum expressed in E. coli from the viewpoint of enzymatic activity. As a result, the present inventors have found that AET derived from S. multivorum having an amino acid sequence in which amino acid residues from the lysine residue at position 2 to the leucine residue at position 18, the histidine residue at position 19, the alanine residue at position 20, the glutamine residue at position 21, the threonine residue at position 22, the alanine residue at position 23 or the alanine residue at position 24 were deleted in the amino acid sequence of SEQ ID NO:2 is localized in the cytosol and is excellent in enzymatic activity as its culture, and have completed the present invention. Based on such findings, the present invention provides the following method for producing the peptide and the method for producing the protein to be used for producing the peptide.

[1] A method for producing a peptide, comprising:

culturing a transformant introduced with an expression vector comprising a polynucleotide encoding any protein selected from the following groups (A)-(E) to prepare a culture; and

mixing said culture with a carboxy component and an amine component to form the peptide from the carboxy component and the amine component,

wherein said group (A) is the group consisting of:

(A18) a protein comprising an amino acid sequence in which amino acid residues from the lysine residue at position 2 to the leucine residue at position 18 are deleted in the amino acid sequence of SEQ ID NO:2;

(A19) a protein comprising an amino acid sequence in which amino acid residues from the lysine residue at position 2 to the histidine residue at position 19 are deleted in the amino acid sequence of SEQ ID NO:2;

(A20) a protein comprising an amino acid sequence in which amino acid residues from the lysine residue at position 2 to the alanine residue at position 20 are deleted in the amino acid sequence of SEQ ID NO:2;

(A21) a protein comprising an amino acid sequence in which amino acid residues from the lysine residue at position 2 to the glutamine residue at position 21 are deleted in the amino acid sequence of SEQ ID NO:2;

(A22) a protein comprising an amino acid sequence in which amino acid residues from the lysine residue at position 2 to the threonine residue at position 22 are deleted in the amino acid sequence of SEQ ID NO:2;

(A23) a protein comprising an amino acid sequence in which amino acid residues from the lysine residue at position 2 to the alanine residue at position 23 are deleted in the amino acid sequence of SEQ ID NO:2; and

(A24) a protein comprising an amino acid sequence in which amino acid residues from the lysine residue at position 2 to the alanine residue at position 24 are deleted in the amino acid sequence of SEQ ID NO:2,

said group (B) is the group consisting of proteins comprising a mutation of one or several amino acid residues that is selected from the group consisting of substitution, deletion, insertion and addition in any protein selected from said group (A), and having a peptide-forming activity,

said group (C) is the group consisting of proteins having 70% or more amino acid sequence identity to any protein selected from said group (A), and having a peptide-forming activity,

said group (D) is the group consisting of proteins encoded by a polynucleotide that hybridizes under a stringent condition with a polynucleotide consisting of a nucleotide sequence complementary to a polynucleotide encoding any protein selected from said group (A), and having a peptide-forming activity, and

said group (E) is the group consisting of proteins encoded by a polynucleotide having 70% or more nucleotide sequence identity to a polynucleotide encoding any protein selected from the group (A), and having a peptide-forming activity.

[2] The method for producing the peptide according to [1], wherein said group (A) is the group consisting of said (A18), (A19), (A20) and (A21).
[3] The method for producing the peptide according to [1] or [2], wherein said transformant is cultured under a temperature condition that is no less than 27° C. but no more than 35° C.
[4] The method for producing the peptide according to any one of [1] to [3], wherein said transformant is derived from Escherichia coli.
[5] The method for producing the peptide according to any one of [1] to [4], wherein the peptide is a dipeptide.
[6] The method for producing the peptide according to any one of claims [1] to [5], wherein the carboxy component is an amino acid ester.
[7] The method for producing the peptide according to any one of [1] to [6], wherein the carboxy component is aspartic acid dimethyl ester, the amine component is phenylalanine, and the peptide is α-L-aspartyl-L-phenylalanine-β-ester.
[8] A method for producing a protein, comprising:

constructing a transformant introduced with an expression vector comprising a polynucleotide encoding any protein selected from the following groups (A)-(E); and

culturing said transformant to express said protein,

wherein said group (A) is the group consisting of:

(A18) a protein comprising the amino acid sequence in which the amino acid residues from the lysine residue at position 2 to the leucine residue at position 18 are deleted in the amino acid sequence of SEQ ID NO:2;

(A19) a protein comprising the amino acid sequence in which the amino acid residues from the lysine residue at position 2 to the histidine residue at position 19 are deleted in the amino acid sequence of SEQ ID NO:2;

(A20) a protein comprising the amino acid sequence in which the amino acid residues from the lysine residue at position 2 to the alanine residue at position 20 are deleted in the amino acid sequence of SEQ ID NO:2;

(A21) a protein comprising the amino acid sequence in which the amino acid residues from the lysine residue at position 2 to the glutamine residue at position 21 are deleted in the amino acid sequence of SEQ ID NO:2;

(A22) a protein comprising the amino acid sequence in which the amino acid residues from the lysine residue at position 2 to the threonine residue at position 22 are deleted in the amino acid sequence of SEQ ID NO:2;

(A23) a protein comprising the amino acid sequence in which the amino acid residues from the lysine residue at position 2 to the alanine residue at position 23 are deleted in the amino acid sequence of SEQ ID NO:2; and

(A24) a protein comprising the amino acid sequence in which the amino acid residues from the lysine residue at position 2 to the alanine residue at position 24 are deleted in the amino acid sequence of SEQ ID NO:2,

said group (B) is the group consisting of proteins comprising a mutation of one or several amino acid residues that is selected from the group consisting of substitution, deletion, insertion and addition in any protein selected from said group (A), and having a peptide-forming activity,

said group (C) is the group consisting of proteins having 70% or more amino acid sequence identity to any protein selected from said group (A), and having the peptide-forming activity,

said group (D) is the group consisting of proteins encoded by a polynucleotide that hybridizes under a stringent condition with a polynucleotide consisting of a nucleotide sequence complementary to a polynucleotide encoding any protein selected from said group (A), and having the peptide-forming activity, and

said group (E) is the group consisting of proteins encoded by a polynucleotide having 70% or more nucleotide sequence identity to a polynucleotide encoding any protein selected from the group (A), and having the peptide-forming activity.

[9] The method for producing the protein according to [8], wherein said transformant is cultured under a temperature condition that is no less than 27° C. but no more than 35° C.
[10] The method for producing the protein according to [8] or [9], wherein said transformant is derived from Escherichia coli.

### Effect of the Invention

According to the present invention, a method for producing a peptide with high efficiency is provided because the peptide is produced using a culture easily solubilized and having a high enzymatic activity per unit amount. In the method for producing the peptide according to the present invention, a culture containing an enzyme is prepared easily and simply, which is advantageous for the method for industrially producing the peptide. An amount of the culture to be added can be reduced because a culture having a high enzymatic activity can be prepared.

### BRIEF DESCRIPTION OF DRAWINGS

FIG. 1 is a view showing a result of SDS-polyacrylamide electrophoresis. A fraction in each lane is as follows. First lane: whole fraction of disrupted microbial cells of pET22b AET-expressing strain. Second lane: supernatant fraction obtained by centrifuging the fraction loaded into the first lane. Third lane: precipitation fraction obtained by centrifuging the fraction loaded into the first lane. Fourth lane: whole fraction of disrupted microbial cells of pET22b n/s21-AET-expressing strain. Fifth lane: supernatant fraction obtained by centrifuging the fraction loaded into the fourth lane. Sixth lane: precipitation fraction obtained by centrifuging the fraction loaded into the fourth lane. Seventh lane: whole fraction of disrupted microbial cells of pET22b pelB21-AET-expressing strain. Eighth lane: supernatant fraction obtained by centrifuging the fraction loaded into the seventh lane. Ninth lane; precipitation fraction obtained by centrifuging the fraction loaded into the seventh lane.

FIG. 2 is views showing relative values of α-L-aspartyl-L-phenylalanine-β-ester forming activity and glucose-6-phosphate dehydrogenase activity when total activity in a cytosol fraction (Cy) and a periplasm fraction (Pe) is 100% (hereinafter, α-L-aspartyl-L-phenylalanine-β-ester may be abbreviated as AMP).

FIG. 3 is a view showing a result of SDS-polyacrylamide electrophoresis. A fraction in each lane is as follows. First lane: whole fraction of disrupted microbial cells of pET22b AET-expressing strain cultured at 25° C. Second lane: supernatant fraction obtained by centrifuging the fraction loaded into the first lane. Third lane: precipitation fraction obtained by centrifuging the fraction loaded into the first lane. Fourth lane: whole fraction of disrupted microbial cells of pET22b n/s21-AET-expressing strain cultured at 25° C. Fifth lane: supernatant fraction obtained by centrifuging the fraction loaded into the fourth lane. Sixth lane: precipitation fraction obtained by centrifuging the fraction loaded into the fourth lane. Seventh lane: whole fraction of disrupted microbial cells of pET22b pelB21-AET-expressing strain cultured at 25° C. Eighth lane: supernatant fraction obtained by centrifuging the fraction loaded into the seventh lane. Ninth lane: precipitation fraction obtained by centrifuging the fraction loaded into the seventh lane.

FIG. 4 is a view showing a result of SDS-polyacrylamide electrophoresis. A fraction in each lane is as follows. First lane: whole fraction of disrupted microbial cells of pET22b AET-expressing strain cultured at 30° C. Second lane: supernatant fraction obtained by centrifuging the fraction loaded into the first lane. Third lane: precipitation fraction obtained by centrifuging the fraction loaded into the first lane. Fourth lane: whole fraction of disrupted microbial cells of pET22b n/s21-AET-expressing strain cultured at 30° C. Fifth lane: supernatant fraction obtained by centrifuging the fraction loaded into the fourth lane. Sixth lane: precipitation fraction obtained by centrifuging the fraction loaded into the fourth lane. Seventh lane: whole fraction of disrupted microbial cells of pET22b pelB21-AET-expressing strain cultured at 30° C. Eighth lane: supernatant fraction obtained by centrifuging the fraction loaded into the seventh lane. Ninth lane: precipitation fraction obtained by centrifuging the fraction loaded into the seventh lane.

FIG. 5 is a view showing a result of SDS-polyacrylamide electrophoresis. A fraction in each lane is as follows. First lane: whole fraction of disrupted microbial cells of pET22b AET-expressing strain cultured at 25° C. Second lane: supernatant fraction obtained by centrifuging the fraction loaded into the first lane. Third lane: precipitation fraction obtained by centrifuging the fraction loaded into the first lane. Fourth lane: whole fraction of disrupted microbial cells of pET22b n/s21-AET-expressing strain cultured at 25° C. Fifth lane: supernatant fraction obtained by centrifuging the fraction loaded into the fourth lane. Sixth lane: precipitation fraction obtained by centrifuging the fraction loaded into the fourth lane. Seventh lane: whole fraction of disrupted microbial cells of pET22b n/s25-AET-expressing strain cultured at 25° C. Eighth lane: supernatant fraction obtained by centrifuging the fraction loaded into the seventh lane. Ninth lane: precipitation fraction obtained by centrifuging the fraction loaded into the seventh lane. Tenth lane: whole fraction of disrupted microbial cells of pET22b n/s26-AET-expressing strain cultured at 25° C. Eleventh lane: supernatant fraction obtained by centrifuging the fraction loaded into the tenth lane. Twelfth lane: precipitation fraction obtained by centrifuging the fraction loaded into the tenth lane. Thirteenth lane: whole fraction of disrupted microbial cells of pET22b n/s27-AET-expressing strain cultured at 25° C. Fourteenth lane: supernatant fraction obtained by centrifuging the fraction loaded into the thirteenth lane. Fifteenth lane: precipitation fraction obtained by centrifuging the fraction loaded into the thirteenth lane.

### BEST MODES FOR CARRYING OUT THE INVENTION

Hereinafter, the embodiments of the present invention will be described. For various gene engineering techniques mentioned below, many standard experimental manuals such as Molecular Cloning: A Laboratory Manual, 3rd edition, Cold Spring Harbor Press (2001) and New Gene Engineering Handbook, revised fourth edition edited by Muramatsu et al., Yodosha (2003) are available, and a person skilled in the art can perform these techniques with reference to these references.

Although the terms used herein are basically used in accordance with standard meanings in chemical, life science and gene engineering fields, parts of the terms used herein will be described below in order to more definitely describe the present invention.

As used herein, the term “enzyme” refers to a protein having an activity to catalyze a chemical reaction.

As used herein, the term “peptide” refers to a compound in which two or more amino acids or derivatives thereof are linked via one or more peptide bonds. Herein, the peptide and a polypeptide are synonymous as the compound.

As used herein, the term “dipeptide” refers to a compound in which two amino acids or derivatives thereof are linked via a peptide bond.

As used herein, the term “tripeptide” refers to a compound in which three amino acids or derivatives thereof are linked via two peptide bonds.

As used herein, the term “oligopeptide” refers to a polypeptide comprising a small number of amino acid residues in its molecule. The oligopeptide is a polypeptide having a low molecular weight. The number of the amino acid residues in the oligopeptide need not be necessarily determined definitely, but is about 2 to 20, or about 2 to 10, or about 2 to 5.

The present invention provides a method for producing a peptide by utilizing an enzymatic reaction. As used herein, the objective peptide to be formed may be expressed simply by “peptide.”

As used herein, the term “peptide-forming activity” refers to an activity to catalyze a reaction of forming a peptide from an amine component and a carboxy component.

As used herein, the terms “polynucleotide” or “nucleic acid” can be DNA, RNA or a hybrid thereof.

As used herein, the term “SEQ ID NO” indicates SEQ ID NO in Sequence Listing unless otherwise specified.

The method for producing the peptide according to the present invention comprises culturing a transformant introduced with an expression vector comprising a polynucleotide encoding a protein having a peptide-forming activity to prepare a culture (culture preparation step), and mixing the culture with a carboxy component and an amine component to form the peptide from the carboxy component and the amine component (reaction step). The method will be sequentially described below along each step.

### 1. Culture Preparation Step

### 1-1. Protein Used in the Present Invention

In the culture preparation step, the transformant introduced with the expression vector comprising the polynucleotide encoding any protein selected from groups (A)-(E) is cultured to obtain the culture.

The group (A) is composed of the group consisting of following (A18) to (A24).

(A18) a protein comprising the amino acid sequence in which the amino acid residues from the lysine residue at position 2 to the leucine residue at position 18 are deleted in the amino acid sequence of SEQ ID NO:2.

(A19) a protein comprising the amino acid sequence in which the amino acid residues from the lysine residue at position 2 to the histidine residue at position 19 are deleted in the amino acid sequence of SEQ ID NO:2.

(A20) a protein comprising the amino acid sequence in which the amino acid residues from the lysine residue at position 2 to the alanine residue at position 20 are deleted in the amino acid sequence of SEQ ID NO:2.

(A21) a protein comprising the amino acid sequence in which the amino acid residues from the lysine residue at position 2 to the glutamine residue at position 21 are deleted in the amino acid sequence of SEQ ID NO:2.

(A22) a protein comprising the amino acid sequence in which the amino acid residues from the lysine residue at position 2 to the threonine residue at position 22 are deleted in the amino acid sequence of SEQ ID NO:2.

(A23) a protein comprising the amino acid sequence in which the amino acid residues from the lysine residue at position 2 to the alanine residue at position 23 are deleted in the amino acid sequence of SEQ ID NO:2.

(A24) a protein comprising the amino acid sequence in which the amino acid residues from the lysine residue at position 2 to the alanine residue at position 24 are deleted in the amino acid sequence of SEQ ID NO:2.

Each protein of (A18) to (A24) may be rephrased as follows in the light of the amino acid sequence of SEQ ID NO:2.

(A18) a protein composed of the amino acid sequence whose amino acid residue at the N terminal end is methionine, subsequently thereto consisting of a full length sequence from the histidine residue at position 19 to the aspartic acid residue at position 619 in the amino acid sequence of SEQ ID NO:2.

(A19) a protein composed of the amino acid sequence whose amino acid residue at the N terminal end is methionine, subsequently thereto consisting of a full length sequence from the alanine residue at position 20 to the aspartic acid residue at position 619 in the amino acid sequence of SEQ ID NO:2.

(A20) a protein composed of the amino acid sequence whose amino acid residue at the N terminal end is methionine, subsequently thereto consisting of a full length sequence from the glutamine residue at position 21 to the aspartic acid residue at position 619 in the amino acid sequence of SEQ ID NO:2.

(A21) a protein composed of the amino acid sequence whose amino acid residue at the N terminal end is methionine, subsequently thereto consisting of a full length sequence from the threonine residue at position 22 to the aspartic acid residue at position 619 in the amino acid sequence of SEQ ID NO:2.

(A22) a protein composed of the amino acid sequence whose amino acid residue at the N terminal end is methionine, subsequently thereto consisting of a full length sequence from the alanine residue at position 23 to the aspartic acid residue at position 619 in the amino acid sequence of SEQ ID NO:2.

(A23) a protein composed of the amino acid sequence whose amino acid residue at the N terminal end is methionine, subsequently thereto consisting of a full length sequence from the alanine residue at position 24 to the aspartic acid residue at position 619 in the amino acid sequence of SEQ ID NO:2.

(A24) a protein composed of the amino acid sequence whose amino acid residue at the N terminal end is methionine, subsequently thereto consisting of a full length sequence from the aspartic acid residue at position 25 to the aspartic acid residue at position 619 in the amino acid sequence of SEQ ID NO:2.

The protein consisting of the amino acid sequence of SEQ ID NO:2 is AET derived from S. multivorum and has a peptide-forming activity. The amino acid sequence of SEQ ID NO:2 may be encoded by multiple nucleotide sequences due to degeneracy of codons. The examples of the nucleotide sequence encoding the amino acid sequence of SEQ ID NO:2 include the nucleotide sequence of SEQ ID NO:1.

The polynucleotide specified by the nucleotide sequence of SEQ ID NO:1 and the protein specified by the amino acid sequence of SEQ ID NO:2 can be isolated from S. multivorum. More specifically, the polynucleotide and protein can be isolated from Sphingobacterium multivorum FERM BP-10163 strain (notation for identification given by the depositor: Sphingobacterium multivorum AJ2458). The bacterial strain specified by the FERM Number has been deposited to International Patent Organism Depositary (IPOD), National Institute of Advanced Industrial Science and Technology (AIST) (Chuo No. 6, Higashi 1-1-1, Tsukuba City, Ibaraki Pref., JP, Postal code 305-8566), and can be obtained with reference to the FERM number. Polynucleotides substantially equivalent to the polynucleotide represented by the nucleotide sequence of SEQ ID NO:1 and proteins substantially equivalent to the protein represented by the amino acid sequence of SEQ ID NO:2 can be isolated by a person skilled in the art from a microorganism belonging to genus Sphingobacterium such as Sphingobacterium multivorum.

In the protein of the group (A), parts or all of the amino acid residues from the lysine residue at position 2 to the alanine residue at position 20 are deleted in the amino acid residues from the methionine residue at position 1 to the alanine residue at position 20, which are estimated to correspond to the signal peptide in the N terminal region of the amino acid sequence of AET derived from S. multivorum (hereinafter, the sequence of the amino acid residues from the methionine residue at position 1 to the alanine residue at position 20 in the amino acid sequence of SEQ ID NO:2, which is deduced to correspond to the signal peptide, may be abbreviated as the signal peptide). The protein of the group (A) has a peptide-forming activity. The protein of the group (A) strongly tends to be localized in the cytosol when expressed in the microbial cell because this protein comprises no signal peptide. The protein of the group (A) is more easily solubilized in an aqueous solution than the protein having the signal peptide. A culture obtained by culturing cells expressing the protein of the group (A) has the enhanced enzymatic activity per unit amount of the culture compared with a culture obtained by culturing cells expressing the protein having the signal peptide. The protein of the group (A) tends to maintain the activity at higher temperature compared with the protein having the signal peptide. Thus, the temperature at which the culture is prepared may be set to be higher, and it is possible to shorten a preparation time of the culture. It is thought that these properties may be associated with the property that the protein of the group (A) is hard to form an inclusion body.

Among the proteins belonging to the group (A), the proteins of (A18), (A19), (A20) and (A21) may be preferable in consideration of the peptide-forming activity and solubilization in a comprehensive manner. More preferably, the proteins of (A19) and (A20) may be suitable for the industrial production of the peptide.

Not only the protein of the group (A) but also protein groups substantially equivalent thereto are used in the method for producing the peptide according to the present invention. The protein groups equivalent to the protein of the group (A) include proteins belonging to any of following groups (B) to (E).

Group (B): a group consisting of proteins comprising a mutation of one or several amino acid residues that is selected from the group consisting of substitution, deletion, insertion and addition in any protein selected from the group (A), and having a peptide-forming activity.

Group (C): a group consisting of proteins having 70% or more amino acid sequence identity to any protein selected from the group (A), and having a peptide-forming activity.

Group (D): a group consisting of proteins are encoded by a polynucleotide that hybridize under a stringent condition with a polynucleotide consisting of a nucleotide sequence complementary to a polynucleotide encoding any protein selected from the group (A), and having a peptide-forming activity.

Group (E): a group consisting of proteins are encoded by a polynucleotide that having 70% or more nucleotide sequence identity to the polynucleotide encoding any protein selected from the group (A), and having a peptide-forming activity.

The protein in each of the groups (B) to (E) is the protein that is different in amino acid sequence (or nucleotide sequence encoding its amino acid sequence) in the range in which a three-dimensional structure or the activity of the protein of the group (A) is not significantly impaired. The protein of the groups (B) to (E) includes proteins corresponding to the proteins of (A18) to (A24) belonging to the group (A).

The protein of the group (B) includes the proteins of the following (B18) to (B24).

(B18) a protein comprising a mutation of one or several amino acid residues that is selected from the group consisting of substitution, deletion, insertion and addition in the protein of (A18), and having a peptide-forming activity.

(B19) a protein comprising a mutation of one or several amino acid residues that is selected from the group consisting of substitution, deletion, insertion and addition in the protein of (A19), and having a peptide-forming activity.

(B20) a protein comprising a mutation of one or several amino acid residues that is selected from the group consisting of substitution, deletion, insertion and addition in the protein of (A20), and having a peptide-forming activity.

(B21) a protein comprising a mutation of one or several amino acid residues that is selected from the group consisting of substitution, deletion, insertion and addition in the protein of (A21), and having a peptide-forming activity.

(B22) a protein comprising a mutation of one or several amino acid residues that is selected from the group consisting of substitution, deletion, insertion and addition in the protein of (A22), and having a peptide-forming activity.

(B23) a protein comprising a mutation of one or several amino acid residues that is selected from the group consisting of substitution, deletion, insertion and addition in the protein of (A23), and having a peptide-forming activity.

(B24) a protein comprising a mutation of one or several amino acid residues that is selected from the group consisting of substitution, deletion, insertion and addition in the protein of (A24), and having a peptide-forming activity.

The protein of the group (C) includes the proteins of the following (C18) to (C24).

(C18) a protein having 70% or more amino acid sequence identity to the protein of (A18), and having a peptide-forming activity.

(C19) a protein having 70% or more amino acid sequence identity to the protein of (A19), and having a peptide-forming activity.

(C20): a protein having 70% or more amino acid sequence identity to the protein of (A20), and having a peptide-forming activity.

(C21) a protein having 70% or more amino acid sequence identity to the protein of (A21), and having a peptide-forming activity.

(C22) a protein having 70% or more amino acid sequence identity to the protein of (A22), and having a peptide-forming activity.

(C23) a protein having 70% or more amino acid sequence identity to the protein of (A23), and having a peptide-forming activity.

(C24) a protein having 70% or more amino acid sequence identity to the protein of (A24), and having a peptide-forming activity.

The protein of the group (D) includes the proteins of the following (D18) to (D24).

(D18) a protein encoded by a polynucleotide that hybridizes under a stringent condition with a polynucleotide consisting of a nucleotide sequence complementary to a polynucleotide encoding the protein of (A18), and having a peptide-forming activity.

(D19) a protein encoded by a polynucleotide that hybridizes under a stringent condition with a polynucleotide consisting of a nucleotide sequence complementary to a polynucleotide encoding the protein of (A19), and having a peptide-forming activity.

(D20) a protein encoded by a polynucleotide that hybridizes under a stringent condition with a polynucleotide consisting of a nucleotide sequence complementary to a polynucleotide encoding the protein of (A20), and having a peptide-forming activity.

(D21) a protein encoded by a polynucleotide that hybridizes under a stringent condition with a polynucleotide consisting of a nucleotide sequence complementary to a polynucleotide encoding the protein of (A21), and having a peptide-forming activity.

(D22) a protein encoded by a polynucleotide that hybridizes under a stringent condition with a polynucleotide consisting of a nucleotide sequence complementary to a polynucleotide encoding the protein of (A22), and having a peptide-forming activity.

(D23) a protein encoded by a polynucleotide that hybridizes under a stringent condition with a polynucleotide consisting of a nucleotide sequence complementary to a polynucleotide encoding the protein of (A23), and having a peptide-forming activity.

(D24) a protein encoded by a polynucleotide that hybridizes under a stringent condition with a polynucleotide consisting of a nucleotide sequence complementary to a polynucleotide encoding the protein of (A24), and having a peptide-forming activity.

The protein of the group (E) includes the proteins of the following (E18) to (E24).

(E18) a protein encoded by a polynucleotide having 70% or more nucleotide sequence identity to a polynucleotide encoding the protein of (A18), and having a peptide-forming activity.

(E19) a protein encoded by a polynucleotide having 70% or more nucleotide sequence identity to a polynucleotide encoding the protein of (A19), and having a peptide-forming activity.

(E20) a protein encoded by a polynucleotide having 70% or more nucleotide sequence identity to a polynucleotide encoding the protein of (A20), and having a peptide-forming activity.

(E21) a protein encoded by a polynucleotide having 70% or more nucleotide sequence identity to a polynucleotide encoding the protein of (A21), and having a peptide-forming activity.

(E22) a protein encoded by a polynucleotide having 70% or more nucleotide sequence identity to a polynucleotide encoding the protein of (A22), and having a peptide-forming activity.

(E23) a protein encoded by a polynucleotide having 70% or more nucleotide sequence identity to a polynucleotide encoding the protein of (A23), and having a peptide-forming activity.

(E24) a protein encoded by a polynucleotide having 70% or more nucleotide sequence identity to a polynucleotide encoding the protein of (A24), and having a peptide-forming activity.

The protein of the groups (B) to (E) retains a peptide-forming activity. It is desirable that the protein of the groups (B) to (E) retains the peptide-forming activity at about a half or more, preferably 80% or more, more preferably 90% or more, still more preferably 95% or more of the peptide-forming activity that the corresponding protein of the group (A) has under the condition at 20° C. at pH 8.5.

The protein of the group (B) includes the protein corresponding to each protein of (A18) to (A24) belonging to the group (A). The number represented by the term “one or several” varies depending on positions and types in the three dimensional structure of the protein with amino acid residues, and for example, denotes 1 to 100, preferably 1 to 70, more preferably 1 to 40, more preferably 1 to 20, more preferably 1 to 10 and still more preferably 1 to 5.

The protein of the group (C) is a protein having 70% or more amino acid sequence identity to any protein selected from the group (A), and having a peptide-forming activity. The amino acid sequence identity is preferably 80% or more, more preferably 85% or more, still more preferably 90% or more, and yet still more preferably 95%, 96%, 97%, 98% or 99% or more. A numerical value showing the identity between the multiple sequences can be calculated by software for sequence analysis. The numerical value of the amino acid sequence identity herein is the numerical value obtained by calculating a marching count as a percentage over full length polypeptide chains and using GENETYX Ver 7.0.9 that is the software of Genetyx Corporation with setup of Unit Size to Compare=2.

The protein of the group (D) is a protein encoded by a polynucleotide that hybridizes under a stringent condition with a polynucleotide consisting of a nucleotide sequence complementary to a polynucleotide encoding any protein selected from the group (A), and having a peptide-forming activity. The “stringent condition” refers to the condition where a so-called specific hybrid is formed whereas no non-specific hybrid is formed. Such a condition is, for example, hybridization in 6×SSC (sodium chloride/sodium citrate) at about 45° C. followed by washing in 0.2×SSC and 0.1% SDS at 50 to 65° C. once or twice or more. Genes that hybridize under such a condition include a gene comprising a stop codon in an internal sequence and a gene in which the activity is lost due to the mutation of an active center, but they can be easily removed by ligating the gene to a commercially available expression vector, expressing the gene in an appropriate host and measuring the enzymatic activity in the expressed product by methods described later.

The protein of the group (E) is a protein encoded by a polynucleotide having 70% or more nucleotide sequence identity to a polynucleotide encoding any protein selected from the group (A), and having a peptide-forming activity. The nucleotide sequence identity is preferably 80% or more, more preferably 85% or more, still more preferably 90% or more, and yet still more preferably 95%, 96%, 97%, 98% or 99% or more. The numerical value showing the identity between the multiple sequences can be calculated by the software for the sequence analysis. The numerical value of the nucleotide sequence identity shown herein is the numerical value obtained by calculating the percentage using full length polynucleotide chains and using GENETYX Ver 7.0.9 that is the software of Genetyx Corporation with setup of Unit Size to Compare=6, pick up location=1.

When the amino acid residue is mutated by substitution, the substitution of the amino acid residue may be conservative substitution. The term “conservative substitution” as used herein refers to that a given amino acid residue is substituted with an amino acid residue having an analogous side chain. Families of the amino acid residues having the analogous side chain are well-known in the art. Examples of such families include amino acids having a basic side chain (e.g., lysine, arginine, histidine), amino acids having an acidic side chain (e.g., aspartic acid, glutamic acid), amino acids having an uncharged polar side chain (e.g., glycine, asparagine, glutamine, serine, threonine, tyrosine, cysteine), amino acids having a nonpolar side chain (e.g., alanine, valine, leucine, isoleucine, proline, phenylalanine, methionine, tryptophan), amino acids having a β position branched side chain (e.g., threonine, valine, isoleucine), amino acids having an aromatic side chain (e.g., tyrosine, phenylalanine, tryptophan, histidine), amino acids having a hydroxyl group (e.g., alcoholic, phenolic)-containing side chain (e.g., serine, threonine, tyrosine), and amino acids having a sulfur-containing side chain (e.g., cysteine, methionine). Preferably, the conservative substitution of the amino acid may be the substitution between aspartic acid and glutamic acid, the substitution between arginine, lysine and histidine, the substitution between tryptophan and phenylalanine, the substitution between phenylalanine and valine, the substitution between leucine, isoleucine and alanine, and the substitution between glycine and alanine.

The protein used in the present invention can be prepared by inserting the polynucleotide encoding the protein which may be obtained by mutagenesis such as site-directed mutagenesis, into an expression vector optionally having a tag sequence such as a sequence for purification. The protein having the amino acid sequence as above may be acquired by conventionally known mutation treatments. Examples of the mutation treatment include a method of treating DNA encoding the protein specified by the amino acid sequence of SEQ ID NO:2 with hydroxylamine in vitro, and a method of treating a bacterium belonging to genus Escherichia retaining DNA encoding that protein with ultraviolet irradiation or a mutating agent such as N-methyl-N′-nitro-N-nitrosoguanidine (NTG) or nitrous acid typically used for artificial mutation.

The mutation as above also includes naturally occurring mutations such as differences due to species or strains of microorganisms. DNA encoding the protein substantially equivalent to the protein specified by the amino acid sequence of SEQ ID NO:2 is obtained by expressing DNA having the mutation as above in appropriate cells and examining the activity of the present enzyme in the expressed product.

### 1-2. Preparation of Culture (Method for Producing Protein)

In the culture preparation step, the culture containing the protein from the above groups (A) to (E) is prepared by producing any protein selected from the above groups (A) to (E) in a transformant and culturing the transformant.

The transformant expressing any protein selected from the groups (A)-(E) can be obtained by constructing an expression vector in which a polynucleotide having the nucleotide sequence encoding the protein is incorporated and introducing the expression vector into an appropriate host. For example, the transformant expressing the protein of (A18) can be obtained by preparing a DNA fragment consisting of the nucleotide sequence in which the nucleotide sequence from the adenine at position 4 to the adenine at position 54 in the polynucleotide sequence of SEQ ID NO:1 are deleted, constructing an expression vector in which this DNA fragment is incorporated and introducing this expression vector into an appropriate host. A transformant also expressing the other protein can be prepared in the same manner.

The expression vector used for introducing a certain DNA into a host can be constructed by inserting the DNA into a desired vector so that the protein encoded by the DNA can be expressed depending on the type of a host for expressing the protein.

As the host for expressing the protein, a cell that is highly proliferative in the cultivation and easily handled is suitable, and a microorganism can be used in general. For example, various prokaryotic cells including cells from bacteria belonging to genus Escherichia, e.g., Escherichia coli, bacteria belonging to genus Corynebacterium, and Bacillus subtilis, and various eukaryotic cells including cells from Saccharomyces cerevisiae, Pichia stipitis and Aspergillus oryzae can be used as preferable microorganisms as the host. E. coli is suitable in the industrial production of the peptide in terms of easiness of cultivation and handling. Further describing E. coli in detail, E. coli to be used can be selected from E. coli K12 strain subspecies, JM109 strain, DH5α strain, HB101 strain, BL21 (DE3) strain, and the like. Methods of performing transformation and methods of selecting the transformants are described in references such as Molecular Cloning: A Laboratory Manual, 3rd edition, Cold Spring Harbor press (2001/01/15).

The method of preparing transformed E. coli and producing a certain enzyme using this will be specifically described below as one example.

For example, a vector such as pUC19, pUC18, pBR322, pHSG299, pHSG298, pHSG399, pHSG398, RSF1010, pACYC177, pACYC184, pMW119, pMW118, pMW219, pMW218, pQE30, or a derivatives thereof may be used. So-called multiple copying types are preferable as the vector. Plasmids having a replication origin derived from ColE1, such as pUC type plasmids, pBR322 type plasmids or derivatives thereof are suitable. The vector of phage DNA may also be utilized as the other vector. Further, the expression vector that comprises a promoter and can express the inserted DNA sequence may be used.

In order to select the transformant, it is preferable that the vector has a marker such as an ampicillin resistant gene. The expression vectors having a strong promoter are commercially available (e.g., pUC types (supplied from TAKARA BIO Inc.), pPRO types (supplied from Clontech), pKK233-2 (supplied from Clontech)) as such plasmids.

The promoter typically used for the production of a heterogeneous protein in E. coli can be used as the promoter, and examples thereof include strong promoters such as T7 promoter, lac promoter, trp promoter, trc promoter, tac promoter, and PR and PL promoters of lambda phage, and T5 promoter.

In order to increase the amount of the produced protein, it may be preferable to ligate a terminator that is a transcription termination sequence to a downstream of a DNA encoding the objective protein. The examples of the terminator include T7 terminator, fd phage terminator, T4 terminator, a terminator in a tetracycline resistant gene, and a terminator in an E. coli trpA gene.

An expression vector can be constructed by ligating a promoter, a gene encoding the objective protein having the peptide-forming activity or a fusion protein of the objective protein with the other protein and any terminator in this order to make a DNA fragment, and further ligating the resulting DNA fragment to the vector DNA using certain restriction enzymes.

A protein of interest is produced by transforming E. coli with the resulting expression vector and culturing the transformed E. coli to express the protein.

The desired protein can be produced on a large scale by culturing and growing the host in an appropriate medium depending on the type of the host. The medium is not particularly limited as long as the host can grow therein, and may be a common medium containing a usual carbon source, a nitrogen source, a phosphorous source, a sulfur source, inorganic ions, and further if necessary organic nutrition sources.

When it is mainly assumed that the host is the microorganism, ingredients to be added in the medium include the following ingredients.

For example, anything can be used as the carbon source as long as the above microorganism can utilize it. Specifically sugars such as glucose, fructose, maltose and amylose, alcohols such as sorbitol, ethanol and glycerol, organic acids such as fumaric acid, citric acid, acetic acid and propionic acid and salts thereof, carbohydrates such as paraffin, and mixtures thereof can be used as the carbon source.

Ammonium salts of inorganic acids such as ammonium sulfate and ammonium chloride, ammonium salts of organic acids such as ammonium fumarate and ammonium citrate, nitrate salts such as sodium nitrate and potassium nitrate, and organic nitrogen compounds such as peptone, yeast extracts, meat extracts and corn steep liquor, and mixtures thereof can be used as the nitrogen source.

In addition, the nutrition sources such as inorganic salts, trace amount metal salts and vitamins used for the common media can be appropriately mixed and used.

The medium such as M9-casamino acid medium and LB medium typically used for culturing E. coli may be used as a production medium. A cultivation condition and a production induction condition can be appropriately selected depending on the marker and the promoter in the vector to be used and the type of the host.

The culture used for the method for producing the peptide according to the present invention is obtained by culturing the transformant as above, and contains any protein selected from the above groups (A) to (E). Examples of a specific form of the culture include the cultured transformant, the medium used for the cultivation, a substance formed by the cultured transformant, and mixtures thereof. For example, a cultured microorganism itself, a medium used for culturing the microorganism, a substance formed by the cultured microorganism, and mixture thereof can be used as the culture when the microorganism is used as the host. A treated microbial cell product may be used as the culture, and a form of the treated microbial cell product include a disrupted microbial cell product, a lysed microbial cell product, and a lyophilized microbial cell product. Further, the culture may be crudely purified to increase a concentration of the protein having the peptide-forming activity.

One embodiment of the method for producing the peptide according to the present invention include an embodiment in which the obtained microbial cell or the mixture containing the same is utilized as the culture. According to the embodiment, cumbersome procedures such as disruption and lysis of the microbial cells is not required because the microbial cell can be directly used. The embodiment is also advantageous in that a possibility of contact of peptide-degrading enzymes exposed by the disruption or lysis of the microbial cell with the peptide as the objective to be produced can be reduced.

A cultivation temperature of the transformant may be controlled depending on the type of the host to be transformed. In one aspect of the present invention, a lower limit of the cultivation temperature can be preferably 27° C. or higher, more preferably 28° C. or higher, and still more preferably 29° C. or higher. Even if the protein such as the protein in the above groups (A) to (E), the signal peptide of which is originally removed, is cultured at the above lower limit temperature or higher, the inclusion body is harder to be formed and the protein is solubilized more easily compared with the protein in which the signal peptide remains (on the other hand, if a protein having the signal peptide is cultured at 27° C. or higher, the inclusion body is easily formed and the protein is easily insolubilized). Generally, the microorganism such as E. coli widely used as the host can be rapidly grown by culturing at higher temperature than the above lower limit. That is, by expressing the protein such as the protein in the above groups (A) to (E), the signal peptide of which is originally removed, it is possible to make the setup of the temperature easy so as to enhance a growth rate of the transformant and enhance a production efficiency of the objective protein having the peptide-forming activity. An upper limit value is appropriately determined in terms of temperature at which the objective protein is not denatured or proliferative property of the host. The upper limit value of the cultivation temperature can be, for example, 55° C. or lower, 40° C. or lower, or 35° C. or lower. As described above, the lower limit and the upper limit of the cultivation temperature can be controlled. In one preferable embodiment of the cultivation temperature, the temperature is controlled to 29° C. to 30° C.

### 2. Reaction Step

The method for producing the peptide according to the present invention comprises mixing the culture prepared as above with a carboxy component and an amine component to form the peptide from the carboxy component and the amine component. The reaction step and the culture preparation step may be carried out simultaneously or separately.

A reaction temperature is, for example, no less than 0° C. but no more than 60° C., and preferably no less than 5° C. but no more than 40° C. The protein used in the present invention is expressed in the state where the signal peptide is originally deleted, and keeps the enzymatic activity even at higher temperature compared with the protein in which signal peptide remains. Thus, the reaction step can be performed at relatively higher temperature and it is possible to set the temperature condition under which the peptide forming reaction is easily promoted, compared with the case of using the protein in which signal peptide remains.

A reaction pH value is, for example, 6.5 to 10.5, and preferably 7.0 to 10.0.

An amount of the culture to be used can be an amount by which an objective effect is exerted (effective amount), and this effective amount is easily determined by a simple preliminary experiment by a person skilled in the art.

One preferable embodiment for performing the peptide forming reaction include an embodiment in which in the condition of 50 mM aspartic acid dimethyl ester hydrochloride, 75 mM L-phenylalanine and 100 mM borate buffer (pH 8.5) at 20° C., 2.2 U of the enzyme (cultured medium) is added to 1 mL of the reaction solution. In order to satisfy this condition, the amount of the microbial cells expressing the enzyme having the signal peptide, and the amount of the microbial cells expressing the enzyme with deletion of the signal peptide were calculated with reference to numerical values in Table 3-1 in following Example 6. According to Table 3-1 in the following Example, AMP forming activity is 2.96 U/mL per OD1 in pET22b AET that is the microbial cell expressing the enzyme having the signal peptide. On the other hand, the AMP forming activity is 4.69 U/mL per OD1 in pET22b n/s21 AET that is one of the microbial cells expressing the enzyme with deletion of the signal peptide. Therefore, 0.74 mL of the microbial cell culture is required per OD1 in pET22b AET, while 0.44 mL of the microbial cell culture is required per OD1 in pET22b n/s21 AET. Thus, 0.30 mL of the microbial cell culture can be reduced in the latter.

The amounts of the microbial cells to be added were compared as follows when 2.2 U of the enzyme (cultured medium) was added to 1 mL of the reaction solution at 25° C. According to Table 3-2 in following Example, the AMP forming activity is 1.15 U/mL per OD1 in pET22b AET that is the microbial cell expressing the enzyme having the signal peptide. On the other hand, the AMP forming activity is 4.58 U/mL per OD1 in pET22b n/s20 AET that is one of the microbial cells expressing the enzyme with deletion of the signal peptide. Therefore, 1.91 mL of the microbial cell culture is required per OD1 in pET22b AET, while 0.48 mL of the microbial cell culture is required per OD1 in pET22b n/s20 AET. Thus, 1.43 mL of the microbial cell culture can be reduced in the latter.

The culture obtained in the above culture preparation step is highly soluble and can produce the high enzymatic activity per unit amount of the culture. Thus, the amount of the culture to be added can be reduced, and it is possible to reduce a production cost. The peptide that is the product of the method of the present invention can be collected, for example, using a filter. Thus, by reducing the amount of the culture to be added, it is possible to reduce a load to the filter, and it is also possible to decrease a replacement frequency of the filter. Therefore, it is also possible from this point to reduce the production cost. A risk that the objective peptide to be produced is degraded by the peptide-degrading enzyme derived from the host can be reduced because the amount of the microbial cell to be added in the reaction system can be reduced by the enhanced enzymatic activity per unit amount of the culture.

When the culture of the microorganism is used, if an enzyme that is not involved in formation of the peptide and degrades the formed peptide is present, it may be more preferable to add a metal protease inhibitor such as ethylenediamine tetracetic acid (EDTA). The amount of the metal protease inhibitor is in the range of 0.1 mM to 300 mM, and preferably 1 mM to 100 mM.

The carboxy component and the amine component are added as substrates to the reaction system for forming the peptide. As used herein, the carboxy component refers to a component donating a carbonyl group (—CO—) in the reaction of forming the peptide bond (—CONH—), and the amine component refers to a component donating an amino group (—NH—) in the reaction of forming the peptide bond (—CONH—).

Any carboxy component may be used as long as the carboxy component can be condensed with the amine component that is another substrate to form the peptide. Examples of the carboxy component include L-amino acid ester, D-amino acid ester, L-amino acid amide, D-amino acid amide, and organic acid ester having no amino group. Not only esters of naturally occurring amino acids but also esters of non-naturally occurring amino acid are exemplified as the amino acid esters. In addition to α-amino acid ester, β-, γ-, and ω-amino acid eaters in which positions of binding the amino group are different are also exemplified as the amino acid esters. Representatives of amino acid esters include methyl ester, ethyl ester, n-propyl ester, iso-propyl ester, n-butyl ester, iso-butyl ester and tert-butyl ester of amino acids.

Any amine component may be used as long as the amine component can be condensed with the carboxy component that is another substrate to form the peptide. Examples of the amine component include L-amino acid, C-protected L-amino acid, D-amino acid, C-protected D-amino acid and amine. Not only naturally occurring amine but also non-naturally occurring amine and derivative thereof are exemplified as amine. Not only naturally occurring amino acids but also non-naturally occurring amino acids and derivative thereof are exemplified as the amino acids. In addition to α-amino acids, β-, γ-, and ω-amino acids in which positions of binding the amino group are different are also exemplified as the amine component.

The concentrations of the carboxy component and the amine component that are starting materials are each 1 mM to 10 M, and preferably 0.05 M to 2 M. It may be more preferable to add the amine component in the amount equivalent to or more than the amount of the carboxy component. When the reaction is inhibited due to the presence of the substrate at high concentration, the substrate may be sequentially added to the reaction so as not to achieve the concentration adjusted to inhibit the reaction.

The method for producing the peptide according to the present invention is suitable for producing various peptides. Examples of the peptides include dipeptides such as α-L-aspartyl-L-phenylalanine-β-methyl ester (i.e., α-L-(β-O-methyl aspartyl)-L-phenylalanine [abbreviation: α-AMP]), L-alanyl-L-glutamine (Ala-Gln), L-alanyl-L-phenylalanine (Ala-Phe), L-phenylalanyl-L-methionine (Phe-Met), L-leucyl-L-methionine (Leu-met), L-isoleucyl-L-methionine (Ile-Met), L-methionyl-L-methionine (Met-Met), L-prolyl-L-methionine (Pro-Met), L-tryptophanyl-L-methionine (Trp-met), L-valyl-L-methionine (Val-Met), L-asparaginyl-L-methionine (Asn-Met), L-cysteinyl-L-methionine (Cys-Met), L-glutamyl-L-methionine (Gln-Met), glycil-L-methionine (Gly-Met), L-seryl-L-methionine (Ser-Met), L-threonyl-L-methionine (Thr-Met), L-tyrosinyl-L-methionine (Tyr-Met), L-aspartyl-L-methionine (Asp-Met), L-arginyl-L-methionine (Arg-Met), L-histidinyl-L-methionine (His-Met), L-lysyl-L-methionine (Lys-Met), L-alanyl-glycine (Ala-Gly), L-alanyl-L-threonine (Ala-Thr), L-alanyl-L-glutamic acid (Ala-Glu), L-alanyl-L-alanine (Ala-Ala), L-alanyl-L-aspartic acid (Ala-Asp), L-alanyl-L-serine (Ala-Ser), L-alanyl-L-methionine (Ala-Met), L-alanyl-L-valine (Ala-Val), L-alanyl-L-lysine (Ala-Lys), L-alanyl-L-asparagine (Ala-Asn), L-alanyl-L-cysteine (Ala-Cys), L-alanyl-L-tyrosine (Ala-Tyr), L-alanyl-L-isoleucine (Ala-Ile), L-arginyl-L-glutamine (Arg-Gln), glycil-L-serine (Gly-Ser), glycil-L-(t-butyl)serine (Gly-Ser (tBu)) and (2S,3R,4S)-4-hydroxylisoleucyl-phenylalanine (HIL-Phe); tripeptides such as L-alanyl-L-phenylalanyl-L-alanine (AFA), L-alanyl-glycil-L-alanine (AGA), L-alanyl-L-histidinyl-L-alanine (AHA), L-alanyl-L-leucyl-L-alanine (ALA), L-alanyl-L-alanyl-L-alanine (AAA), L-alanyl-L-alanyl-glycine (AAG), L-alanyl-L-alanyl-L-proline (AAP), L-alanyl-L-alanyl-L-glutamine (AAQ), L-alanyl-L-alanyl-L-tyrosine (AAY), glycil-L-phenylalanyl-L-alanine (GFA), L-alanyl-glycil-glycine (AGG), L-threonyl-glycyl-glycine (TGG), glycyl-glycyl-glycine (GGG) and L-alanyl-L-phenylalanyl-glycine (AFG); tetrapeptides such as glycil-glycil-L-phenylalanyl-L-methionine (GGFM); and pentapeptides such as L-tyrosyl-glycil-glycil-L-phenylalanyl-L-methionine (YGGFM).

In one preferable embodiment of the method for producing the peptide according to the present invention, the carboxy component is aspartic acid dimethyl ester, the amine component is phenylalanine, and the peptide to be formed is aspartyl-phenylalanine. More specifically, the method for producing the peptide according to the present invention is suitable as the method for producing α-L-aspartyl-L-phenylalanine-β-methyl ester (i.e., α-L-(β-O-methyl aspartyl)-L-phenylalanine [abbreviation: α-AMP]). α-AMP is an important intermediate for producing α-L-aspartyl-L-phenylalanine-α-methyl ester (product name: aspartame) that has large demands as a sweetener.

### EXAMPLES

The present invention will be described in more detail with reference to the following Examples, but the present invention is not limited thereto.

### Example 1

### Construction of Expression Plasmid

<1-1> Construction of Plasmid Expressing AET Substituted with Signal Peptide pelB

PCR of 30 cycles (30 seconds at 94° C., one minute at 52° C. and 2 minutes at 68° C.) was performed using the expression plasmid pSF_Sm_Aet (WO2006/075486 A1) comprising full length AET derived from S. multivorum as a template, and a primer represented by the sequence of SEQ ID NO:3 (a primer designed to amplify from the codon corresponding to the 21st amino acid from the N terminus of AET derived from S. multivorum subsequent to a NcoI recognition sequence) as a sense primer, and a primer represented by the nucleotide sequence of SEQ ID NO:4 (a primer designed to amplify from a stop codon of AET subsequent to a XhoI recognition sequence) as an antisense primer. Subsequently, the resulting PCR product was digested with NcoI/XhoI. After agarose gel electrophoresis, the objective DNA of about 1.8 kb was collected from agarose gel and ligated to a NcoI-XhoI site of pET22b (Novagen). Their nucleotide sequences were confirmed, and correct one was designated as pET22b pelB21-AET. The plasmid pET22b pelB21-AET is the plasmid expressing “AET substituted with the signal peptide pelB”, in which the signal peptide included in full length AET derived from S. multivorum is substituted with the signal peptide pelB. The signal peptide pelB is the signal peptide derived from Erwinia cartovora.

<1-2> Construction of Plasmid Expressing AET with Deletion of Signal Peptide

PCR of 30 cycles (30 seconds at 94° C., one minute at 52° C. and 2 minutes at 68° C.) was performed using the expression plasmid pSF_Sm_Aet (WO2006/075486 A1) comprising full length AET derived from S. multivorum as the template, and a primer represented by the nucleotide sequence of SEQ ID NO:5 (a primer designed to amplify from the codon corresponding to the 21st amino acid from the N terminus of AET derived from S. multivorum subsequent to a NdeI recognition sequence) as the sense primer, and a primer represented by the nucleotide sequence of SEQ ID NO:4 as the antisense primer. Subsequently, the resulting PCR product was treated with NdeI/XhoI. After agarose gel electrophoresis, the objective DNA of about 1.8 kb was collected from agarose gel and ligated to a NdeI-XhoI site of pET22b. Their nucleotide sequences were confirmed, and correct one was designated as pET22b n/s21-AET. The plasmid pET22b n/s21-AET is the plasmid expressing AET in which the amino acid residues from the lysine residue at position 2 to the alanine residue at position 20 in the amino acid sequence of SEQ ID NO:2 were deleted, i.e., AET in which the signal peptide included in full length AET derived from S. multivorum was deleted.

### <1-3> Construction of Plasmid Expressing AET

A plasmid expressing AET having the signal peptide derived from S. multivorum was also constructed as a control expression plasmid. PCR of 30 cycles (30 seconds at 94° C., one minute at 52° C. and 2 minutes at 68° C.) was performed using the expression plasmid pSF_Sm_Aet (WO2006/075486 A1) comprising full length AET derived from S. multivorum as the template, and a primer represented by the nucleotide sequence of SEQ ID NO:6 (a primer designed to amplify from the codon corresponding to the second amino acid from the N terminus of AET derived from S. multivorum subsequent to a NdeI recognition sequence) as the sense primer, and a primer represented by the nucleotide sequence of SEQ ID NO:4 as the antisense primer. Subsequently, the resulting PCR product was digested with NdeI/XhoI. After agarose gel electrophoresis, the objective DNA of about 1.9 kb was collected from agarose gel and ligated to the NdeI-XhoI site of pET22b. Their nucleotide sequences were confirmed, and correct one was designated as pET22b AET. The plasmid pET22b AET is the plasmid expressing full length AET derived from S. multivorum, i.e., AET including the wild type signal peptide.

### Example 2

### Expression of AET in E. coli

E. coli BL21 (DE3) transformed with three expression plasmids prepared in Example 1, i.e., pET22b AET, pET22b pelB21-AET and pET22b n/s21-AET were designated as pET22b AET strain, pET22b pelB21-AET strain and pET22b n/s21-AET strain, respectively. pET22b AET strain, pET22b pelB21-AET strain or pET22b n/s21-AET strain was precultured in LB agar medium containing 100 μg/mL of ampicillin at 20° C. for 16 hours. Subsequently, one loopful of the precultured strain expressing the enzyme was inoculated to a medium (4 g/L of glycerol, 24 g/L of yeast extract, 12 g/L of peptone, 2.3 g/L of potassium dihydrogen phosphate, 12.5 g/L of dipotassium hydrogen phosphate, 20 mL/L of Solution 1, 50 ml/L of Solution 2 and 1 mL/L of Solution 3) containing 100 μg/mL of ampicillin in a normal test tube, and cultured at 20° C. at 150 reciprocations/minute for 40 hours to perform the main cultivation, and the cultured microbial cells were obtained (Solutions 1 to 3 are the solutions attached to Overnight Express Autoinduction System 1 (Novagen)).

### Example 3

### Measurement of AET Activity

Each cultured microbial cell obtained in Example 2 was suspended in 100 mM borate buffer (pH 8.5) containing 50 mM aspartic acid dimethyl ester hydrochloride and 75 mM L-phenylalanine, and the mixture was reacted at 20° C. The product was quantified by high performance liquid chromatography (HPLC) shown below, and an AET-forming activity was calculated. An amount of the cultured microbial cell to be added was 2% (v/v).

### (HPLC)

Column: Inertsil ODS-3 (GL Science), eluant: aqueous solution of 100 mM phosphoric acid (pH 2.1), 13% acetonitrile, flow rate: 1.0 mL/minute, column temperature: 40° C., and detection: 210 nm.

Results of the measurements are shown in Table 1. The activity per cultured medium to form AMP (α-aspartyl-L-phenylalanine-β-ester) was confirmed to be enhanced in the AET-expressing strain in which the amino acid residues from the lysine residue at position 2 to the alanine residue at position 20 had been deleted in the amino acid residues from the methionine residue at position 1 to the alanine residue at position 20 which are deduced to be the signal peptide in the amino acid sequence of SEQ ID NO:2 (pET22b n/s21-AET strain), compared with the control strain (pET22b AET strain). On the other hand, the activity per cultured medium in the AET-expressing strain in which the signal peptide had been substituted with the PelB signal peptide (p22ETb pelB21-AET strain) was almost equivalent to that in the control strain.

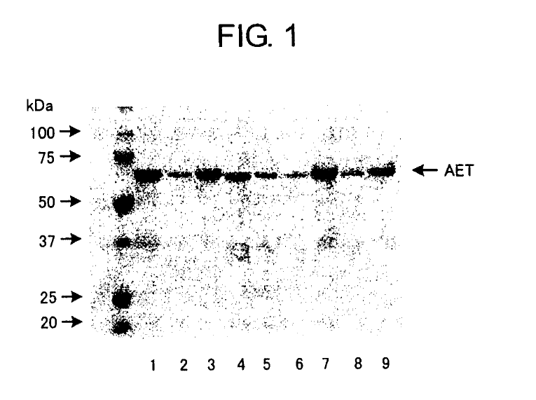

A whole fraction obtained by collecting microbial cells from each cultured medium and disrupting the microbial cells by sonication, a centrifuged supernatant fraction and a centrifuged precipitation fraction were developed on SDS-polyacrylamide gel electrophoresis, and the results are shown in FIG. 1. The amount of AET in the centrifuged supernatant was increased whereas the amount of AET in the centrifuged precipitation fraction was decreased in pET22b n/s21 AET strain, compared with pET22b AET strain and pET22b pelB21 AET strain. That is, it was found that AET in which the amino acid residues from the lysine residue at position 2 to the alanine residue at position 20 had been deleted in the amino acid residues from the methionine residue at position 1 to the alanine residue at position 20 which are deduced to be the signal peptide in the amino acid sequence of SEQ ID NO:2 became easily soluble, thereby enhancing the activity per microbial cells in the cultured medium.

TABLE 1

| | AMP-forming activity (U/ml) per OD1 |
|---|---|
| pET22b AET | 2.9 |
| pET22b pelB21-AET | 3.0 |
| pET22b n/s21-AET | 4.3 |

### Example 4

### Localization of AET

The cultured microbial cells obtained in Example 2 were fractionated into a periplasm fraction and a cytosol fraction by an osmotic shock method using a 25 g/dL sucrose solution. The microbial cells in the 25 g/dL sucrose solution were immersed in a solution of 20 mM Tris-Cl (pH 8.0), and a supernatant obtained by centrifuging this solution was used as the periplasm fraction. The cytosol fraction was obtained by resuspending this centrifuged precipitation and disrupting the cells by the sonication.

In order to confirm that the cytosol was separated, the activity of glucose-6-phosphate dehydrogenase known to be present in the cytosol fraction was used as an indicator. Its activity was measured by adding an appropriate amount of the enzyme to a reaction solution of 1 mM glucose-6-phosphate, 0.4 mM NADP, 10 mM MgSO₄, 50 mM Tris-Cl (pH 8.0) at 30° C. and measuring the formation of NADPH by absorbance at 340 nm.

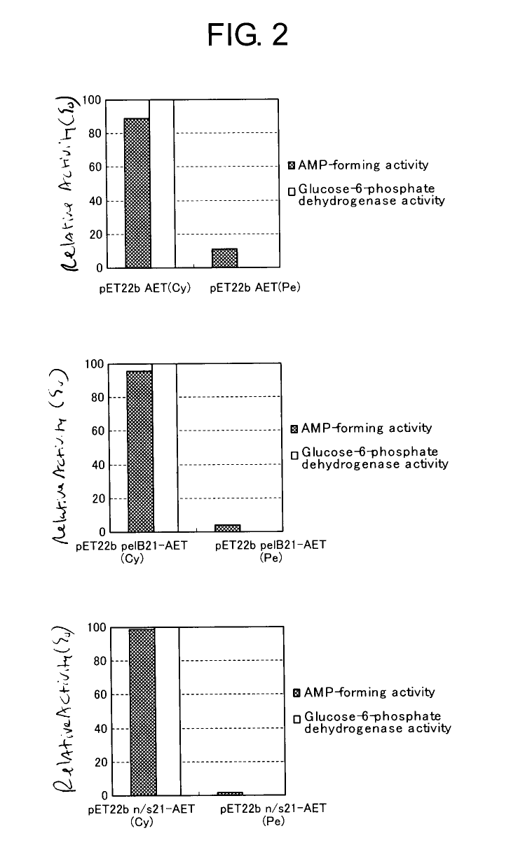

Relative values of the activity when total of the activity in the cytosol fraction (Cy) and the activity in the periplasm fraction (Pe) was 100% are shown in FIG. 2. That the activity of glucose-6-phosphate dehydrogenase is not contaminated in the periplasm fraction indicates that the periplasm fraction is not contaminated in the cytosol fraction. In the total activity, about 90%, about 96% and about 99% were recovered in the cytosol fractions from pET22b-AET strain, ppET22b pelB21-AET strain and pET22b n/s21-AET strain, respectively. That is, AET derived from S. multivorum was predicted to be the enzyme in the periplasm from its amino acid sequence, but AET was not translocated to the periplasm and was expressed in the cytosol fraction although the signal sequence in the N terminal region was cleaved when AET was expressed in E. coli. Also, AET in which the amino acid residues from the lysine residue at position 2 to the alanine residue at position 20 had been deleted in the amino acid residues from the methionine residue at position 1 to the alanine residue at position 20 which are deduced to be the signal peptide in the amino acid sequence of SEQ ID NO:2 was easily solubilized, and exhibited the higher activity per microbial cells in the cultured medium.

### Example 5

### Examination of Cultivation Temperature

It was found that AET became easily soluble by deleting the signal peptide. Thus, it was expected that AET with deletion of the signal sequence had the activity even at higher cultivation temperature. Thus, pET22b-AET strain, pET22b pelB21-AET strain and pET22b n/s21-AET strain were cultured at 25° C. or 30° C. in the method described in Example 2, and the activity per microbial cells in the cultured medium was measured in the method described in Example 3.

The results are shown in Table 2-1 (cultivation temperature at 20° C.), Table 2-2 (cultivation temperature at 25° C.) and Table 2-3 (cultivation temperature at 30° C.). By elevating the cultivation temperature from 20° C. to 25° C., the activity per microbial cells in the cultured medium was reduced to about 40% in pET22b-AET strain and pET22b pelB21-AET strain, while about 86% of the activity was kept in pET22b n/s21-AET strain. At cultivation temperature of 30° C., the activity was further reduced, but the activity per volume of the cultured medium was the highest in pET22b n/s21-AET strain.

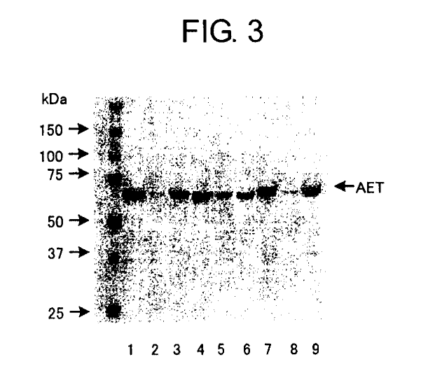

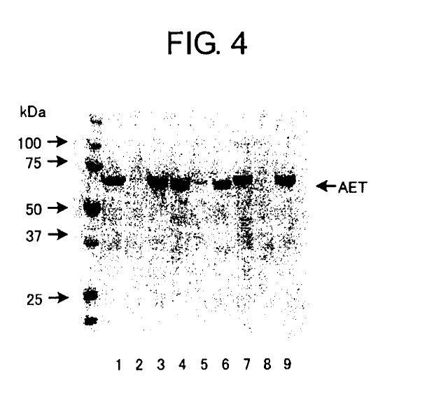

Subsequently, a whole fraction obtained by collecting the microbial cells from the cultured medium at 25° C. and 30° C. and disrupting them by the sonication, a centrifuged supernatant fraction and a centrifuged precipitation fraction obtained by centrifuging the whole fraction were developed on SDS-polyacrylamide gel electrophoresis. The results are shown in FIG. 3 and FIG. 4. A greater amount of AET was confirmed in the centrifuged supernatant fraction (solubilized fraction) from pET22b n/s21-AET strain cultured at 25° C. compared with pET22b AET strain and pET22b pelB21-AET strain (FIG. 3). In the cultivation at 30° C., no AET was confirmed in the centrifuged supernatant fraction from pET22b AET strain and pET22b pelB21-AET strain, but AET was confirmed in the centrifuged supernatant fraction from pET22b n/s21-AET strain (FIG. 4). That is, it was revealed that pET22b n/s21-AET strain with deletion of the signal peptide had the activity at higher cultivation temperature compared with pET22b AET strain and pET22b pelB21-AET strain having the signal peptide.

TABLE 2-1

| Cultured at 20° C. | AMP-forming activity (U/ml) per OD1 |
|---|---|
| pET22b AET | 2.96 |
| pET22b pelB21-AET | 3.87 |
| pET22b n/s21-AET | 4.69 |

TABLE 2-2

| Cultured at 25° C. | AMP-forming activity (U/ml) per OD1 |
|---|---|
| pET22b AET | 1.15 |
| pET22b pelB21-AET | 1.53 |
| pET22b n/s21-AET | 4.01 |

TABLE 2-3

| Cultured at 30° C. | AMP-forming activity (U/ml) per OD1 |
|---|---|
| pET22b AET | 0.33 |
| pET22b pelB21-AET | 0.59 |
| pET22b n/s21-AET | 0.63 |

### Example 6

### Optimization of Starting Amino Acid

A centrifuged supernatant fraction was obtained by collecting the microbial cells from the cultured medium of pET22b AET strain, disrupting them by the sonication and centrifuging them. An N terminal amino acid sequence of AET in the centrifuged supernatant fraction was analyzed, and about 32% thereof was glutamine. These were deduced to be those in which the amino acid residues from the methionine residue at position 1 to the alanine residue at position 20 had been cleaved in the amino acid sequence of SEQ ID NO:2, as predicted from their amino acid sequences. Meanwhile, about 14% aspartic acid, about 11% alanine, and about 9% histidine were also detected, and thus, it was likely to be cleaved at other sites. Thus, for the purpose of optimization of the N terminus, expression plasmids with deletion of various signal sequence were constructed in the methods shown below.

<Construction of AET Expression Plasmids with Deletion of Signal Peptide>

PCR of 30 cycles (30 seconds at 94° C., one minute at 52° C. and 2 minutes at 68° C.) was performed using the expression plasmid pSF_Sm_Aet (WO2006/075486 A1) comprising full length AET derived from S. multivorum as the template, and a primer represented by the nucleotide sequence of SEQ ID NOS:7 to 14 (a primer designed to amplify from the codon corresponding to the 19th, 20th, 22nd, 23rd, 24th, 25th, 26th or 27th amino acid from the N terminus of AET derived from S. multivorum subsequent to the NdeI recognition sequence) as a sense primer, and a primer represented by the nucleotide sequence of SEQ ID NO:4 as an antisense primer.

Subsequently, the resulting PCR product was treated with NdeI/XhoI. After agarose gel electrophoresis, the DNA of about 1.8 kb was collected from agarose gel and ligated to the NdeI-XhoI site of pET22b (Novagen). Their nucleotide sequences were confirmed, and correct ones were designated as pET22b n/s19-AET (deletion from the lysine residue at position 2 to the leucine residue at position 18 in the amino acid sequence of SEQ ID NO:2), pET22b n/s20-AET (deletion from the lysine residue at position 2 to the histidine residue at position 19 in the amino acid sequence of SEQ ID NO:2), pET22b n/s22-AET (deletion from the lysine residue at position 2 to the glutamine residue at position 21 in the amino acid sequence of SEQ ID NO:2), pET22b n/s23-AET (deletion from the lysine residue at position 2 to the threonine residue at position 22 in the amino acid sequence of SEQ ID NO:2), pET22b n/s24-AET (deletion from the lysine residue at position 2 to the alanine residue at position 23 in the amino acid sequence of SEQ ID NO:2), pET22b n/s25-AET (deletion from the lysine residue at position 2 to the alanine residue at position 24 in the amino acid sequence of SEQ ID NO:2), pET22b n/s26-AET (deletion from the lysine residue at position 2 to the aspartic acid residue at position 25 in the amino acid sequence of SEQ ID NO:2), and pET22b n/s27-AET (deletion from the lysine residue at position 2 to the serine residue at position 26 in the amino acid sequence of SEQ ID NO:2).

Subsequently, E. coli BL21 (DE3) transformed with each expression plasmid prepared above was cultured in the method described in Example 2, and the activity per cultured medium was measured according to the method described in Example 3.

The results of measuring the AMP-forming activity are shown in Table 3-1 (cultivation temperature at 20° C.), Table 3-2 (cultivation temperature at 25° C.) and Table 3-3 (cultivation temperature at 30° C.). It was found that pET22b n/s20-AET strain had the higher activity per cultured medium than pET22b n/s21-AET strain at cultivation temperature of 25° C. and 30° C.

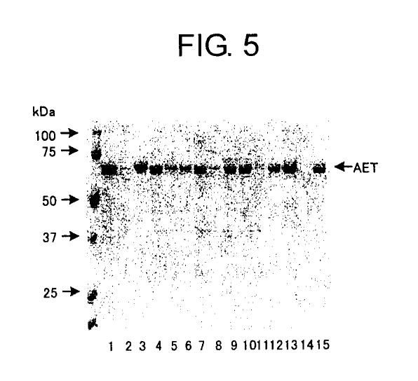

Subsequently, a whole fraction obtained by collecting the microbial cells from the cultured medium at 25° C. and disrupting them by the sonication, a centrifuged supernatant fraction and a centrifuged precipitation fraction obtained by centrifuging the whole fraction were developed on SDS-polyacrylamide gel electrophoresis. The result is shown in FIG. 5. When a 5th lane, a 8th lane, a 11th lane and a 14th lane were compared, the longer the deleted region from the N terminus was, the more the tendency to decrease the AET amount in the centrifuged supernatant was observed. On the other hand, when a 6th lane, a 9th lane, a 12th lane and a 15th lane were compared, the longer the deleted region from the N terminus was, the more the tendency to increase the AET amount in the centrifuged precipitation was observed. From these results, that the longer the deleted region from the N terminus was, the AMP-forming activity per cultured medium was lowered was deduced to be due to not the reduction of the expressed amount but the formation of the inclusion body.

TABLE 3-1

| Cultured at 20° C. | AMP-forming activity (U/ml) per OD1 |
|---|---|
| pET22b AET | 2.96 |
| pET22b n/s19-AET | 4.78 |
| pET22b n/s20-AET | 3.80 |
| pET22b n/s21-AET | 4.69 |
| pET22b n/s22-AET | 3.83 |
| pET22b n/s23-AET | 3.78 |
| pET22b n/s24-AET | 3.47 |
| pET22b n/s25-AET | 3.75 |
| pET22b n/s26-AET | 3.22 |
| pET22b n/s27-AET | 1.31 |

TABLE 3-2

| Cultured at 25° C. | AMP-forming activity (U/ml) per OD1 |
|---|---|
| pET22b AET | 1.15 |
| pET22b n/s19-AET | 4.14 |
| pET22b n/s20-AET | 4.58 |
| pET22b n/s21-AET | 4.01 |
| pET22b n/s22-AET | 2.65 |
| pET22b n/s23-AET | 2.77 |
| pET22b n/s24-AET | 2.31 |
| pET22b n/s25-AET | 2.39 |
| pET22b n/s26-AET | 1.19 |
| pET22b n/s27-AET | 0.17 |

TABLE 3-3

| Cultured at 30° C. | AMP-forming activity (U/ml) per OD1 |
|---|---|
| pET22b AET | 0.33 |
| pET22b n/s19-AET | 0.59 |
| pET22b n/s20-AET | 1.04 |
| pET22b n/s21-AET | 0.63 |
| pET22b n/s22-AET | 0.21 |
| pET22b n/s23-AET | 0.14 |
| pET22b n/s24-AET | 0.20 |
| pET22b n/s25-AET | 0.09 |
| pET22b n/s26-AET | 0.06 |
| pET22b n/s27-AET | 0.01 |

1. A method for producing a peptide, comprising:
culturing a transformant introduced with an expression vector comprising a polynucleotide encoding any protein selected from the following groups (A)-(E) to prepare a culture; and
mixing said culture with a carboxy component and an amine component to form the peptide from the carboxy component and the amine component,
wherein said group (A) is the group consisting of:
(A18) a protein comprising the amino acid sequence in which the amino acid residues from the lysine residue at position 2 to the leucine residue at position 18 are deleted in the amino acid sequence of SEQ ID NO:2;
(A19) a protein comprising the amino acid sequence in which the amino acid residues from the lysine residue at position 2 to the histidine residue at position 19 are deleted in the amino acid sequence of SEQ ID NO:2;
(A20) a protein comprising the amino acid sequence in which the amino acid residues from the lysine residue at position 2 to the alanine residue at position 20 are deleted in the amino acid sequence of SEQ ID NO:2;
(A21) a protein comprising the amino acid sequence in which the amino acid residues from the lysine residue at position 2 to the glutamine residue at position 21 are deleted in the amino acid sequence of SEQ ID NO:2;
(A22) a protein comprising the amino acid sequence in which the amino acid residues from the lysine residue at position 2 to the threonine residue at position 22 are deleted in the amino acid sequence of SEQ ID NO:2;
(A23) a protein comprising the amino acid sequence in which the amino acid residues from the lysine residue at position 2 to the alanine residue at position 23 are deleted in the amino acid sequence of SEQ ID NO:2; and
(A24) a protein comprising the amino acid sequence in which the amino acid residues from the lysine residue at position 2 to the alanine residue at position 24 are deleted in the amino acid sequence of SEQ ID NO:2,
said group (B) is the group consisting of proteins comprising a mutation of one or several amino acid residues that is selected from the group consisting of substitution, deletion, insertion and addition in any protein selected from said group (A), and having a peptide-forming activity,
said group (C) is the group consisting of proteins having 70% or more amino acid sequence identity to any protein selected from said group (A), and having a peptide-forming activity,
said group (D) is the group consisting of proteins encoded by a polynucleotide that hybridizes under a stringent condition with a polynucleotide consisting of a nucleotide sequence complementary to a polynucleotide encoding any protein selected from said group (A), and having a peptide-forming activity, and
said group (E) is the group consisting of proteins encoded by a polynucleotide having 70% or more nucleotide sequence identity to a polynucleotide encoding any protein selected from the group (A), and having a peptide-forming activity.

2. The method for producing the peptide according to claim 1, wherein said group (A) is the group consisting of said (A18), (A19), (A20) and (A21).

3. The method for producing the peptide according to claim 1 or 2, wherein said transformant is cultured under a temperature condition that is no less than 27° C. but no more than 35° C.

4. The method for producing the peptide according to any one of claims 1 to 3, wherein said transformant is derived from Escherichia coli.

5. The method for producing the peptide according to any one of claims 1 to 4, wherein the peptide is a dipeptide.

6. The method for producing the peptide according to any one of claims 1 to 5, wherein the carboxy component is an amino acid ester.

7. The method for producing the peptide according to any one of claims 1 to 6, wherein the carboxy component is aspartic acid dimethyl ester, the amine component is phenylalanine, and the peptide is α-L-aspartyl-L-phenylalanine-β-ester.

8. A method for producing a protein, comprising:
constructing a transformant introduced with an expression vector comprising a polynucleotide encoding any protein selected from the following groups (A)-(E); and
culturing said transformant to express said protein,
wherein said group (A) is the group consisting of:
(A18) a protein comprising the amino acid sequence in which the amino acid residues from the lysine residue at position 2 to the leucine residue at position 18 are deleted in the amino acid sequence of SEQ ID NO:2;
(A19) a protein comprising the amino acid sequence in which the amino acid residues from the lysine residue at position 2 to the histidine residue at position 19 are deleted in the amino acid sequence of SEQ ID NO:2;
(A20) a protein comprising the amino acid sequence in which the amino acid residues from the lysine residue at position 2 to the alanine residue at position 20 are deleted in the amino acid sequence of SEQ ID NO:2;
(A21) a protein comprising the amino acid sequence in which the amino acid residues from the lysine residue at position 2 to the glutamine residue at position 21 are deleted in the amino acid sequence of SEQ ID NO:2;
(A22) a protein comprising the amino acid sequence in which the amino acid residues from the lysine residue at position 2 to the threonine residue at position 22 are deleted in the amino acid sequence of SEQ ID NO:2;
(A23) a protein comprising the amino acid sequence in which the amino acid residues from the lysine residue at position 2 to the alanine residue at position 23 are deleted in the amino acid sequence of SEQ ID NO:2; and
(A24) a protein comprising the amino acid sequence in which the amino acid residues from the lysine residue at position 2 to the alanine residue at position 24 are deleted in the amino acid sequence of SEQ ID NO:2,
said group (B) is the group consisting of proteins comprising a mutation of one or several amino acid residues that is selected from the group consisting of substitution, deletion, insertion and addition in any protein selected from said group (A), and having a peptide-forming activity,
said group (C) is the group consisting of proteins having 70% or more amino acid sequence identity to any protein selected from said group (A), and having the peptide-forming activity,
said group (D) is the group consisting of proteins encoded by a polynucleotide that hybridizes under a stringent condition with a polynucleotide consisting of a nucleotide sequence complementary to a polynucleotide encoding any protein selected from said group (A), and having the peptide-forming activity, and
said group (E) is the group consisting of proteins encoded by a polynucleotide having 70% or more nucleotide sequence identity to a polynucleotide encoding any protein selected from the group (A), and having the peptide-forming activity.

9. The method for producing the protein according to claim 8, wherein said transformant is cultured under a temperature condition that is no less than 27° C. but no more than 35° C.

10. The method for producing the protein according to claim 8 or 9, wherein said transformant is derived from Escherichia coli.
